(19) United States
(12) Patent Application Publication
(10) Pub. No.: US 2024/0090825 A1
(43) Pub. Date: Mar. 21, 2024

(54) SYSTEM AND METHOD FOR EXTRACTION OF JOINT-SPECIFIC MOVEMENT CAPACITY AND MOTION SIGNATURE FROM IMAGING

(71) Applicant: The Cleveland Clinic Foundation, Cleveland, OH (US)

(72) Inventors: Ahmet Erdemir, Chagrin Falls, OH (US); Neda Abdollahi Nohouji, Austin, TX (US); Benjamin Landis, Fort Worth, TX (US)

(21) Appl. No.: 18/226,698

(22) Filed: Jul. 26, 2023

Related U.S. Application Data
(60) Provisional application No. 63/392,599, filed on Jul. 27, 2022.

(51) Int. Cl. A61B 5/00; A61B 5/055; A61B 5/11; A61B 6/00; G16H 50/20; G16H 50/50

(52) U.S. Cl. CPC A61B 5/4514; A61B 5/055; A61B 5/1121; A61B 5/4528; A61B 5/4851; A61B 6/505; A61B 6/5217; G16H 50/20; G16H 50/50

(57) ABSTRACT

A method of imaging and analyzing the joint of a patient, such as a knee, involves acquiring a three-dimensional data of the joint of the patient, reconstructing an articular contact geometry of the joint based on the 3D data, determining a tissue quality on articular contact geometry based on the 3D data, determining movement capacity of the joint based on the reconstructed articular contact geometry; and determining a motion signature of the joint based on the reconstructed articular contact geometry and the tissue quality.

### CROSS-REFERENCE TO RELATED APPLICATIONS

The present application claims the benefit of U.S. Provisional Patent Application No. 63/392,599 filed Jul. 27, 2022, the entirety of which is incorporated by reference herein.

### STATEMENT REGARDING FEDERALLY SPONSORED RESEARCH OR DEVELOPMENT

This invention was made with government support under EB024573 and EB025212 awarded by the National Institutes of Health. The government has certain rights in the invention.

### BACKGROUND

In the United States, there are more than 10 million visits to clinics every year because of musculoskeletal joint injury and pathology, with the knee as a major joint of concern. Further, osteoarthritis impacts more than 27 million in the United States. Due to this, more than 790,000 total knee arthroplasty and more than 100,000 anterior cruciate ligament (ACL) reconstruction surgeries are performed each year.

Each knee has unique mechanics that affect mobility, capacity to withstand injury, pathological degradation, and recovery after an intervention. These mechanics can be characterized by metrics that describe different biomechanical states and functions of the knee. For example, “movement capacity” (the permissible bounds of motion) is an inherent property and an indicator of biomechanical stability and performance, i.e., how much the joint can move; and “motion signature” (habitual movement patterns) is an indicator of physical activity preferences and motion styles, i.e., how the joint does move. However, determination of these metrics is mostly a research endeavor that can require sophisticated biomechanical experimentation or laborious (and arguably unproven) physics-based computational modeling.

While it is possible to acquire experimental information in vivo, testing is highly burdensome for the analyst and the patient. Moreover, in vivo testing generally only focuses on select activities, and thus does not provide an indication of preferred movement patterns of a subject. Therefore, most experimentation is done in vitro using robotics, which provides comprehensive, albeit cumbersome examination of joint movement bounds. Yet, it is not possible to measure preferred movement patterns as joint loading that dictates motion is assumed. Computational modeling can rely on knee-specific anatomy but their development and utilization requires additional information such as tissue mechanical properties, and assumed loading and boundary conditions. The model outputs then are highly susceptible to each modeler's decisions, and manual labor is common. Further, solutions only consider selected, and mostly generic, loading cases that may not have correspondence to preferred movement intensity and style. As a result, current experimentation and modeling strategies are neither practical nor comprehensive for determination of joint-specific movement capacity and motion signature.

### BRIEF SUMMARY

According to one example of the present disclosure, a method comprises acquiring three-dimensional (3D) data of a joint; reconstructing an articular contact geometry of the joint based on the 3D data; and determining movement capacity of the joint based on the reconstructed articular contact geometry, or determining a tissue quality on articular contact geometry based on the 3D data and determining a motion signature of the joint based on the reconstructed articular contact geometry and the tissue quality.

In various embodiments of the above example, the joint is a musculoskeletal joint; the joint is an artificial joint; reconstructing the articular contact geometry comprises: segmenting bones of the joint based on the 3D data, determining subchondral regions of bone surface geometry of the joint based on the segmented bones and the 3D data, and determining articular contact surfaces based on the subchondral regions; the method comprises determining the movement capacity of the joint, wherein determining the movement capacity of the joint comprises determining combinations of contact points between opposing articular contact surfaces of the reconstructed articular contact geometry, wherein each determined combination of contact points corresponds to a pose and orientation of the joint; determining the movement capacity or the motion signature of the joint comprises excluding determined combinations of contact points in which opposing articular contact surfaces penetrate each other beyond a predetermined threshold; each of the opposing articular contact surfaces have corresponding contact points, and alignment of the corresponding contact points is constrained based on geometric relationships between contact points of opposing articular surfaces; the method comprises determining the tissue quality and determining the motion signature, wherein determining the motion signature comprises: mapping the determined tissue quality on the articular contact geometry, and estimating joint rotations and translations based on contact point sets of opposing articular contact surfaces; the method further comprises: excluding estimated joint rotations and translations based on thresholds of the determined tissue quality mapped to the articular contact geometry at the contact points; the joint rotations and translations are estimated based on locations of local peaks of the determined tissue quality mapped to the articular geometry as contact point candidates, and based on a cumulative tissue quality across contact points of each joint rotation and translation; the method further comprises: segmenting cartilage of the joint based on the 3D data, and generating surface geometries of cartilage based on the segmented cartilage, wherein reconstructing the articular contact geometry comprises generating articular surfaces based on articulating portions of cartilage determined from the segmented cartilage and generated surface geometries of the cartilage; determining the movement capacity or motion signature comprises: estimating joint rotations and translations based on contact point sets of opposing articular contact surfaces, and excluding joint rotations and translations in which cartilage surfaces penetrate each other beyond a predetermined threshold; the joint is a knee and the method further comprises: segmenting menisci tissue of the knee based on the 3D data, and generating surface geometry of menisci based on the segmented menisci, wherein reconstructing the articular contact geometry comprises generating articular surfaces based on the segmented menisci and generated surface geometries of the menisci; determining the movement capacity or the motion signature comprises: estimating joint rotations and translations based on contact point sets of opposing articular contact surfaces, and excluding joint rotations and translations in which opposing articular contact surfaces and menisci penetrate each other beyond a predetermined threshold, the method further comprises determining ligament insertions by: segmenting ligament insertion footprints on joint bones based on the 3D data, and identifying a centroid of ligament insertion based on the segmented ligament insertion footprints, or identifying elevated tissue intensity regions on the joint bones and estimating ligament insertion footprints based on the identified elevated tissue intensity regions; determining the movement capacity or motion signature comprises: estimating joint rotations and translations based on contact point sets of opposing articular contact surfaces, determining a length of a ligament for the estimated joint rotations and translations based on the estimated ligament insertion footprints, determining a statistical distribution of the determined ligament lengths across a plurality of joint rotations and translations, and excluding joint rotations and translations in which ligament lengths exceed a predetermined threshold; the 3D data of the joint is of an unloaded configuration of the joint; the 3D data of the joint is of a loaded configuration of the joint; and/or the method comprises: generating a report of the determined movement capacity and/or the determined motion signature, outputting the report, and diagnosing or treating a patient based on the determined movement capacity or the determined motion signature.

According to one example of the present disclosure, a method comprises: acquiring computed tomography (CT) or magnetic resonance imaging (MRI) three-dimensional (3D) data of a knee of a patient; segmenting a tibia and a femur of the knee based on the 3D data; generating bone surface geometries of the tibia and the femur based on the segmentation and the 3D data; determining articular contact geometries from bone surface geometries of the tibia and femur; determining sets of contact point pairs between the tibia and the femur based on the articular contact geometries; estimating tibiofemoral poses and orientations of the knee based on the determined contact pair sets; determining movement capacity of the knee based on estimated tibiofemoral poses and orientations; determining a bone intensity of the tibia and femur based on the 3D data; mapping a bone quality of the tibia and the femur on the articular contact geometries of the tibia and femur based on the determined bone intensities of the tibia and femur, respectively; estimating tibiofemoral poses and orientations of the knee based on the determined contact pair sets at elevated levels of mapped bone quality; determining a motion signature of the knee based on the estimated tibiofemoral poses and orientations of the knee; and diagnosing or treating the patient based on the determined movement capacity and motion signature of the knee.

### BRIEF DESCRIPTIONS OF THE SEVERAL VIEWS OF THE DRAWING

FIG. 1 is a flowchart of an example method of the present disclosure.

FIG. 2 illustrates an example cross-sectional image of a knee from three-dimensional (3D) image data acquired using computed tomography (CT).

FIG. 3A is a flowchart of an example method of determining movement capacity of a joint according to the present disclosure.

FIG. 3B illustrates an example method of geometric model reconstruction.

FIG. 3C illustrates an example method of determining a database of contact point combinations.

FIG. 3D illustrates a method of reducing the database of joint translations and rotations.

FIG. 4A illustrates reconstructed bone and articular contact geometries.

FIG. 4B illustrates articular contact surfaces as meshes and an example contact point combination thereon.

FIGS. 4C and 4D illustrate an example contact point combination before and after alignment, respectively.

FIG. 4E illustrates an example joint kinematic configuration.

FIG. 4F illustrates reconstructed cartilage, meniscus, and ligament geometries.

FIGS. 4G, 4H, 4I, 4J and 4K illustrate representations of joint movement capacity in a database including medial translation, anterior translation, superior translation, varus-valgus, and internal rotation across flexion, respectively.

FIG. 5A is a flowchart of an example method of determining motion signature of a joint according to the present disclosure when a prior database of joint rotations and translations is available.

FIG. 5B is a flowchart of an example method of determining motion signature of a joint according to the present disclosure when a prior database of joint rotations and translations is not available.

FIG. 6A illustrates geometries of subchondral bone regions on femur and tibia with an overlaid bone quality heat map.

FIG. 6B illustrates local peaks of bone intensity regions on articular contact geometries as candidate contact points for alignment between opposing surfaces.

FIG. 6C illustrates multiple examples of joint kinematic configurations after alignment of high bone intensity regions.

FIG. 7 illustrates a block diagram of an example system for the delivery of joint-specific movement capacity and motion signature from 3D imaging according to the present disclosure.

### DETAILED DESCRIPTIONS OF THE DRAWINGS

Considering the above, present delivery of healthcare, particularly relating to the knee, is not personalized. To improve the personalization, it is desirable to speedily and consistently understand the mechanical potential of an individual subject's knee joint in relation to the joint's functional environment, which is quantified by the determination of knee-specific movement capacity and motion signature as biomarkers. The present disclosure thus relates to systems and methods for comprehensive and individualized evaluation of a joint's movement bounds and its habitual movement pattern. Further the disclosure may be applied to implants and the evaluation thereof (e.g., determining a proper implant for a patient).

While the present disclosure refers to the knee as an example joint, it should be understood that the present disclosure can be applied to any joint, including artificial joint implants. Further, any relevant tissue quality can be utilized where tissue information that is to be mapped on articular contact surfaces. Still further, data can be obtained from any mechanism, such design, drafting, drawing, or imaging, such as computed tomography (CT), magnetic resonance imaging (MM), or the like.

Generally, the above-described determined metrics including movement capacity and motion signature (and their reports) can support diagnosis, prognosis, calculation of risk scores, and guidance for surgical planning. For example, the metrics make it possible to determine which patients have less stable knees, habitual movements that may put them at risk of injury or pathology, and like diagnostic and prognostic determinations. Still further, the metrics can be used during surgery or other clinical interventions. Still further, the metrics may be used to determine appropriate clinical treatments and interventions based on predicted outcomes and likelihoods of success for each possible treatment and intervention for the individual patient's knee.

For example, the metrics can be used to reconstruct an idealized biomechanical state and function of a patient's knee as a target for planning and evaluation of interventions. In anterior cruciate ligament (ACL) surgery, this knowledge can be a personalized reference goal for regaining knee mobility. In total knee arthroplasty, the metrics can be utilized not only to construct pre-surgical movement metrics of the operated knee but also the unoperated contralateral knee, which is commonly used as a reference for anatomy but not for mechanics. Analysis of an implant geometry can quantify the movement capacity offered by the design. In osteoarthritis, the metrics can be utilized for monitoring disruptions in movement capacity and movement patterns as a function of morphological changes.

Briefly, processes for determination of movement capacity and motion signature for the knee, in particular the tibiofemoral joint, are described herein. The system and methods described in the present disclosure implement sequences of determinations, starting with obtaining three dimensional (3D) volumetric data (e.g., medical imaging data), from which databases of movement capacity and motion signature based on six degrees of freedom of relative position and orientation between femur and tibia bones are generated. The database of movement capacity represents all possible kinematic configurations of the tibiofemoral joint, based on constraints imposed by articular contact geometries and when desired, cartilage, meniscus, and ligament geometries. The database of motion signature includes tibiofemoral kinematic configurations that are constrained by alignment of high bone intensity locations on femoral and tibial articular contact geometries, thus represent joint pose and orientations that are frequently loaded.

Determination of movement capacity starts with segmentation of bones (e.g., femur and tibia for the knee joint) to generate bone and articular contact geometries (discretized surfaces) from 3D data. Depending on the embodiment, medical imaging, such as computed tomography or magnetic resonance imaging, can be used as a source of the 3D data. Identification of subchondral regions of the bones permits determination of articular contact geometries, which are then divided into four regions: medial and lateral sides of the femoral and tibial articular contact surfaces. Each point in each of these regions can be used as a contact point candidate. A database of contact point pairs can then be generated for each bone by combining points on the medial side with points on the lateral side; and a database of contact point sets is generated by combining each contact pair on the tibia with each contact pair on the femur.

For each database entry between tibial contact point pairs and femoral contact point pairs, relative position and orientation between femur and tibia (tibiofemoral joint kinematic configuration) is calculated. Determination of the joint's six degrees of freedom is based on a minimization of the distance between medial contact points (one on femur, the other on tibia) and lateral contact points (one on femur, the other on tibia), and maximization of the alignment of contact normals at contact point locations (for the medial side and for the lateral side). This can be achieved, for example, by optimization algorithms based on rigid contact constraints of two articulating bodies. Executing optimization for each database entry results in a plurality of tibiofemoral joint kinematic configurations. This resulting database of joint rotations and translations represents the movement capacity of the joint afforded by articular contact geometries.

The resulting database representing movement capacity can be reduced by removing data entries where penetrations between opposing bone surfaces and between opposing articular surfaces are above a set threshold. When cartilage segmentation is performed and cartilage geometries are available, they can be used to determine articular contact geometries from the surface geometries of superficial, free ends of the cartilage. The database can be further reduced by removing data entries (e.g., sets of joint rotations and translations) where penetration between opposing cartilage surfaces at that joint kinematic configuration exceeds a set threshold. When meniscus segmentation is performed and meniscus geometries are available, the database can be further reduced. Menisci are affixed to tibia and penetrations between meniscal and femoral articular contact surfaces are calculated. A data entry can be excluded when penetration at that joint kinematic configuration exceeds a set threshold. The resulting database then represents the movement capacity of the joint afforded by meniscus function. When ligament geometries (insertion and origins) are identified from 3D data, the database can be further reduced to determine movement capacity of the joint afforded by ligament function. For each data entry, length of each ligament is determined at that joint kinematic configuration. A data entry can be excluded when the length of any of the ligaments exceeds a set threshold. Thresholds for each ligament may be determined by the statistical distribution of ligament lengths for the whole input database of joint rotations and translations.

Ancillary methods can also be implemented for determining movement capacity. For example, image segmentation and geometry generation can follow any available state-of-the-art approaches. For the knee, coordinate systems on the femur and tibia can be established using conventional methods such as using anatomical landmarks or data fits to bone geometries to determine joint coordinate system origins and axes. The kinematic configuration of the knee can then be described by six degrees of freedom joint translations and rotations such as flexion-extension, internal-external rotation, varus-valgus (also called abduction-adduction), anterior-posterior translation, medial-lateral translation, and compression-distraction (also called superior-inferior translation). The database for movement capacity can be ranked by flexion angle to determine movement capacity as a function of flexion such as mean and standard deviation of each degree of freedom at a set flexion angle.

Generation of tibiofemoral joint kinematic configuration database relies on large scale computations. The number of contact pairs on each bone side (medial vs lateral) and the combinatorial correspondence of contact locations between bones (femoral vs tibial) increase the required number of optimizations to determine joint rotations and translations that are compatible with input contact point locations. This burden can be reduced by conducting computations selectively, based on geometric knowledge of contact point pairs on each bone side. For a plausible contact between bones, the distance between medial and lateral contact points of tibia may be equal to the distance between medial and lateral contact points of femur. Similarly, the angle between medial and lateral contact point normals of tibia should be equal to the angle between medial and lateral contact point normals of femur. These constraints can be used to reduce the number of contact point sets passed to the optimization by removing those that exceed set thresholds in matching of opposing contact point pairs' distance and angle metrics.

Determination of joint kinematic configuration is generalizable to situations where contact between the articulating surfaces is a single contact location on each body (instead of two, i.e., one on medial and another on lateral sides of femur and tibia). In this case, optimization aligns the contact points and the contact normals of opposing surfaces to determine joint translations and two of the joint rotations (to align normals). In following, one of the bodies can be rotated incrementally (e.g., one degree at a time) around the contact normal to generate a database of joint kinematic configurations encompassing all possible solutions of the remaining joint rotation (e.g., 0° to 360°).

Determination of motion signature also uses bone and articular contact geometries to generate database of tibiofemoral joint kinematic configurations from contact point candidates. Classification of a joint pose and orientation as part of the motion signature database relies on a tissue quality metric that is mapped on the articular contact surface. Bone quality for each point on articular contact surfaces can be determined, for example, by the average of computed tomography image intensity across a kernel volume attached to the articular surface point and oriented below the surface of a subchondral region. High bone quality regions are indicators of frequent and high mechanical loading and thus their alignment across opposing articular contact surfaces denote frequented joint kinematic configurations where joint loads are transmitted. When a previously determined database of joint rotations and translations exist (e.g., as part of movement capacity calculations), determination of motion signature is the reduction of the database based on bone intensity metrics at contact points corresponding to a given joint kinematic configuration. For the tibiofemoral joint, aggregate bone intensity index can be determined based on medial femoral and tibial contact points as well as lateral femoral and tibial contact points.

In following, the mean of medial and lateral geometric means provides a bone intensity index for the joint kinematic configuration. Joint kinematics database entries that have bone intensity index below a set threshold are removed from the database. The remaining set of joint rotations and translations represents a motion signature of the knee. The ancillary methods described for determining movement capacity can also be useful for determining motion signature, including establishing and using joint coordinate systems, reporting as a function of flexion and summarizing as mean and standard deviation of each degree of freedom at a set flexion angle.

Without a prior database of joint poses and orientations, determination of motion signature is possible by the identification of a reduced set of candidate contact points for efficient calculation of joint kinematic configurations. The bone intensity maps on articulating surfaces are analyzed to label points that are local peaks. For the tibiofemoral joint, the collection of these labeled points provides a reduced cohort of contact point locations on medial and lateral regions of opposing articular contact surfaces (compared to all possible contact point locations). Optimization using combinations of these (e.g., first across medial and lateral regions of each bone, and then across contact pairs of femur and tibia), results in joint kinematics configurations corresponding to motion signature. If desired, an aggregate bone intensity index can be determined, and a threshold can be implemented for each joint kinematic configuration, allowing ranking or further reduction of the database.

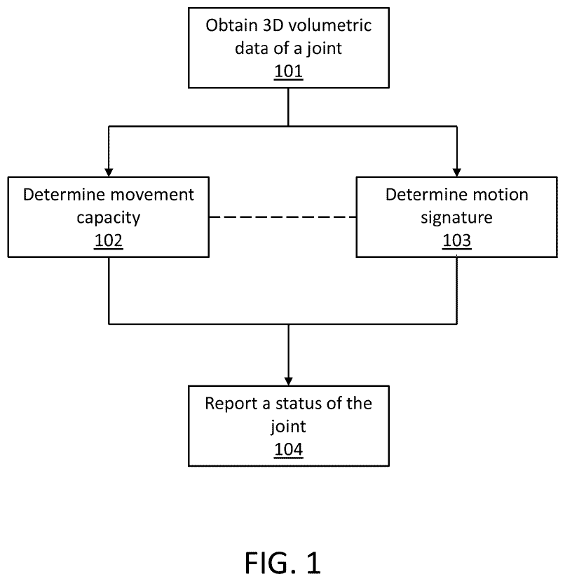

Turning now to the figures, FIG. 1 illustrates an example method for determining a personalized quantified analysis of a joint. Briefly as seen therein, the method first includes obtaining 3D volumetric data of a joint 101. This data may be obtained, for example, by 3D clinical imaging of a joint. In addition or alternatively, 3D volumetric data may be obtained from design, drafting, drawing, or like techniques that do not necessarily involve imaging.

The acquisition of 3D volumetric data of the joint 101 is followed by the steps of determining movement capacity of the joint 102 and determining the motion signature of the joint 103. Determining the movement capacity and motion signature 102, 103 may be performed additionally or alternatively to each other. Further, these determinations 102, 103 may be performed in any order, but as will be discussed in later sections, there may be overlapping aspects between them. Thus, certain aspects of method steps 102 and 103 may be performed in parallel. Using the metrics determined in steps 102 and 103, a step 104 of reporting a status of a joint may be carried out. It is also contemplated that in certain embodiments, the process can be completed while omitting or altering steps while achieving substantially the same results.

As mentioned above, the 3D imaging may performed by any modality, such as computed topography (CT), magnetic resonance imaging (MRI), or the like, which produce 3D volumetric data. The resulting 3D data of the joint can provide a full field view of the knee joint. For example, the volumetric data can be used to generate 3D models, 3D representative images, 2D images (e.g., cross-sectional slices of a volume), and the like. The 3D data may be obtained of an unloaded or loaded joint. After the 3D image is obtained 101, any desired pre-processing steps to prepare the image for clinical use, such as filtering, adjusting and the like, may be performed.

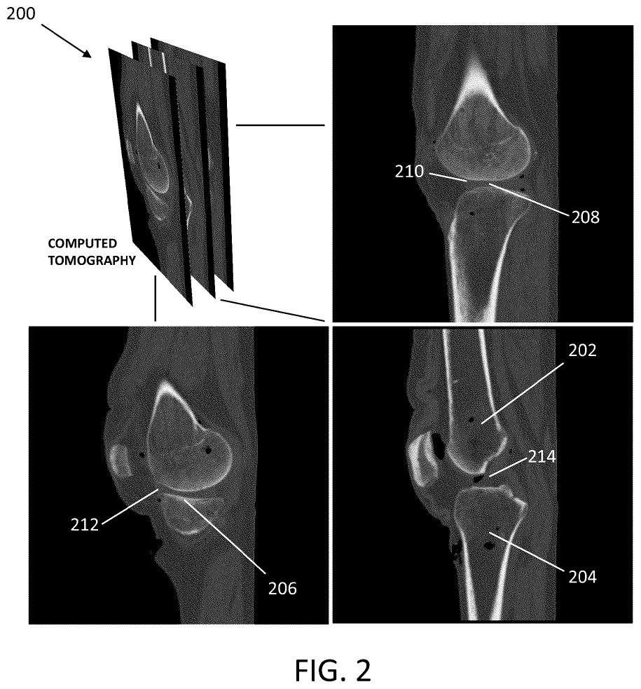

A sample CT scan that may be used in accordance with the systems and process steps described herein is shown in FIG. 2. More particularly, FIG. 2 illustrates an example image generated from 3D volumetric data of a knee 200, obtained from a CT scan. For reference, the CT scan of the knee 200 illustrates the femur 202 and the tibia 204 of the example knee. Further, the CT scan of the knee 200 shows subchondral regions with high bone intensity 206, articulation regions 208, cartilage 210, the meniscus 212, and ligaments 214.

From the 3D volumetric data of the knee 200, a quality metric of a bone may be determined, such as bone intensity. Processing to determine the bone quality metrics can be performed on any portion or all of the 3D volumetric data. Herein, “bone intensity” can refer to the brightness of a voxel of the 3D volumetric data. Generally, a higher brightness is correlated to a higher bone density at a point represented by the voxel, and it is understood that voxel intensity may be a relative value to the brightness of other voxels. Bone intensity may be represented by discrete data points tied to individual voxels, for example represented by a value ranging from 0 to 1. These intensities may be relative values, such as scaled values to each other, as absolute intensities may vary between machines. In FIG. 2, a subchondral high intensity region 206 is shown to be illuminated relative to other areas of the 3D volumetric data of the knee 200. It follows that the high intensity bone region 206 correlates to a region of the tibia with a high mineral bone density.

Other bone quality metrics that may be determined include, for example, bone thickness or other radiomics metrics. Additionally, through methods that will be described herein, articular contact geometries can be derived from the 3D volumetric data. High bone intensity regions (e.g. regions of high bone density), can indicate regions of the knee that are mechanically loaded or stressed. These high-intensity regions can indicate certain tibiofemoral poses and provide information for improving the dataset of contact pairs and for determining a knee-motion signature, such as described in the determination of movement capacity and motion signature 102, 103, respectively.

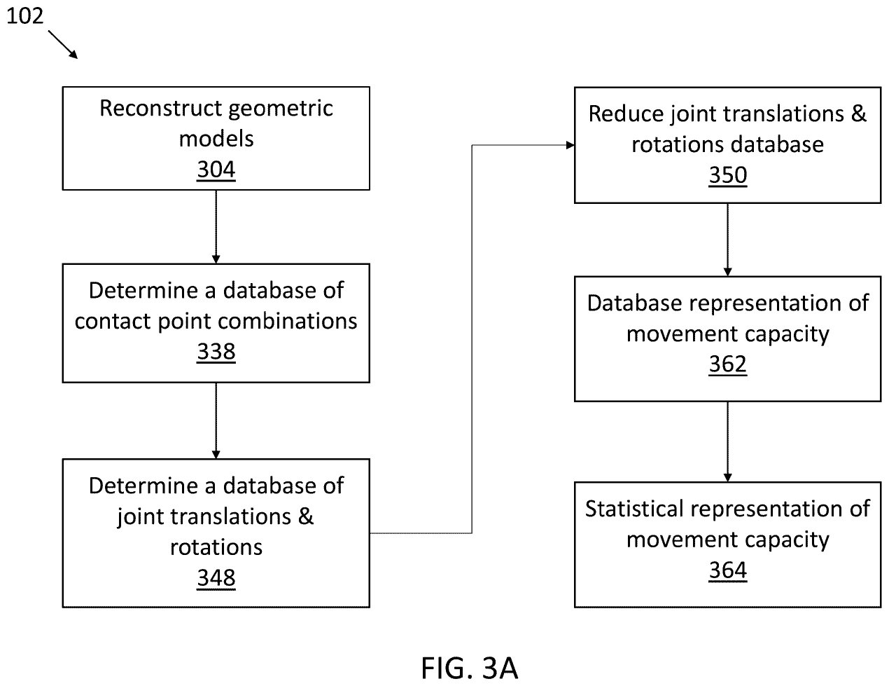

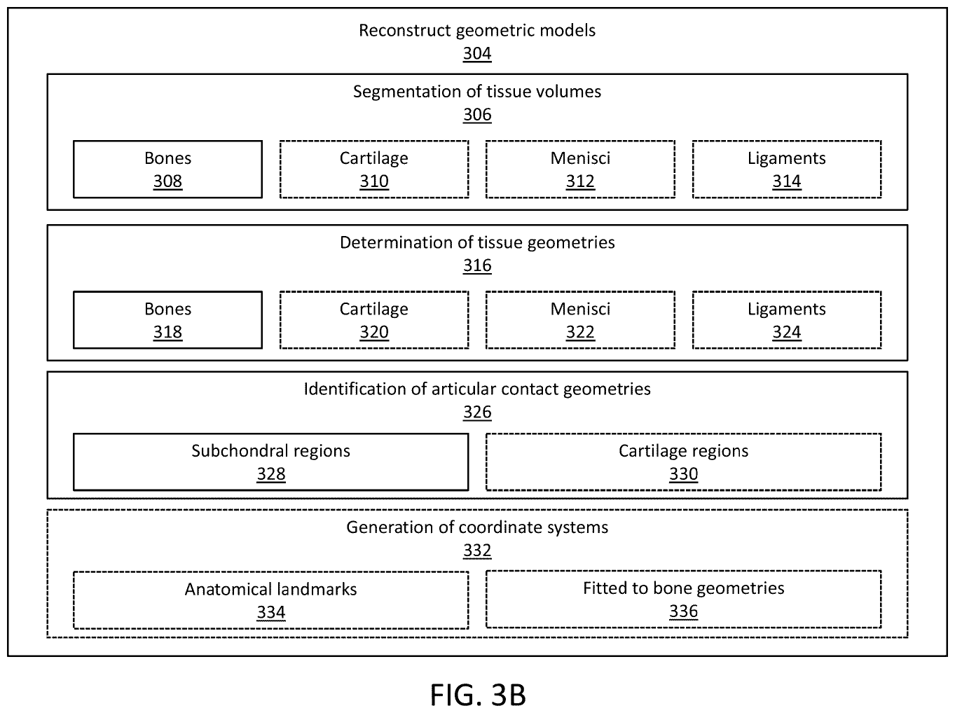

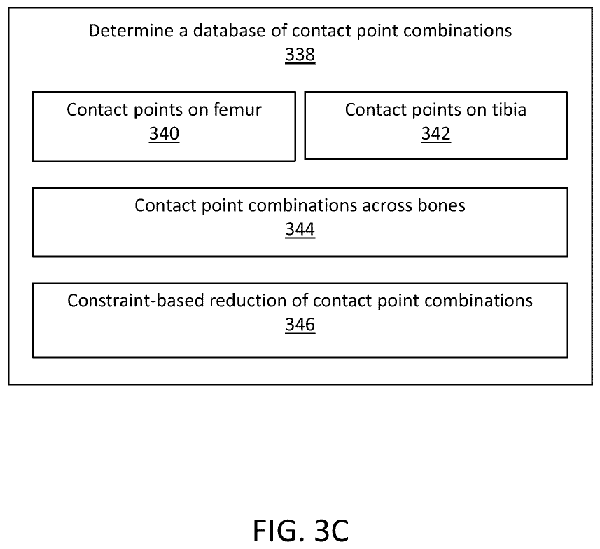

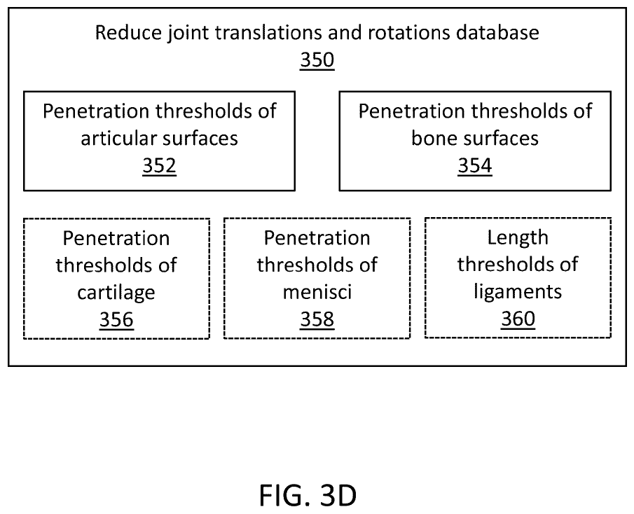

A flowchart illustrating an example method of determining movement capacity 102 is illustrated in FIG. 3A. According to the embodiment of FIG. 3A, determining movement capacity 102 includes of a first step of reconstructing geometric models 304, including articular contact geometry from the 3D volumetric data. Then, sets of possible contact pairs between articular surfaces are determined 338, using the geometric models from step 304. A database of candidate contact points obtained in step 338 is then used to determine a database of joint rotation and translations 348. For example, this may be done by determining kinematic configuration of single contact joints by aligning single contact points from the respective articular surfaces. Based on the aligned contact points, joint translations may be determined where contact points are coincident and where two joint rotations align with contact normals of the articular surfaces. The possible remaining joint rotations may be determined by, beginning for example at an angle of 0 degrees in the joint coordinate plane, rotating one or both of the joint bodies incrementally around the contact normal of the articular surface to, for example, 360 degrees in the coordinate plane. The database of joint rotations and translations obtained in step 348 may be reduced by various optional methods 350. A resulting population set of joint rotations and translations 362 can then be statistically represented as movement bounds 364.

As illustrated in FIG. 3B, the articular contact geometry reconstruction 304 may involve the segmentation of tissue volumes 306, the determination of tissue surface geometry 316, the identification of articular contact geometry 326, and the generation of coordinate systems 332. Segmentation of tissue volumes 306 may include segmentation of bones 308, cartilage 310, menisci 312, and ligaments 314. Any method of segmentation is acceptable, including automated methods achieved through machine learning. Alternatively, segmentation may be manually performed by clinicians, skilled medical technicians and the like. Generation of tissue geometries may optionally include those of bones 318, cartilage 320, menisci 322, and ligaments 324. When available, these geometries may be used to augment analysis in further steps, i.e., they can provide additional contact constraints that will improve the population of contact point pair sets.

The segmentation of tissue volume 306 can involve analyzing sections of the 3D data and grouping pixels into clusters, regions, and/or segments correlated to specific bones. In doing this, portions of the 3D data associated with different bones can be isolated for analysis. For example, in certain embodiments, the tibia and the femur are segmented, but other bone volumes of interest may be segmented when applied to joints other than a knee.

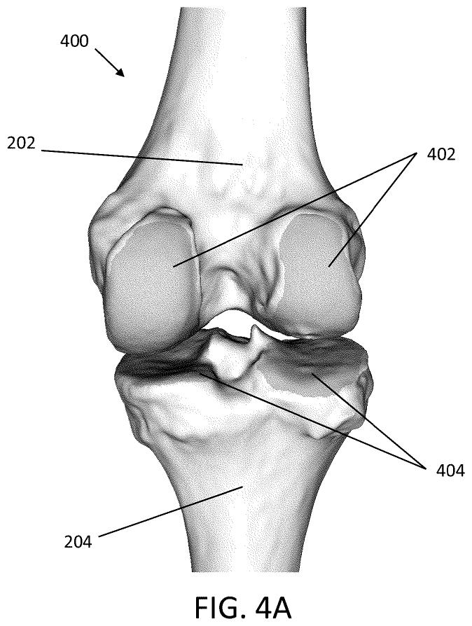

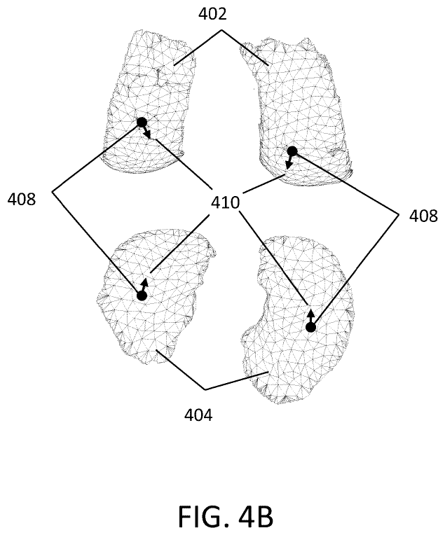

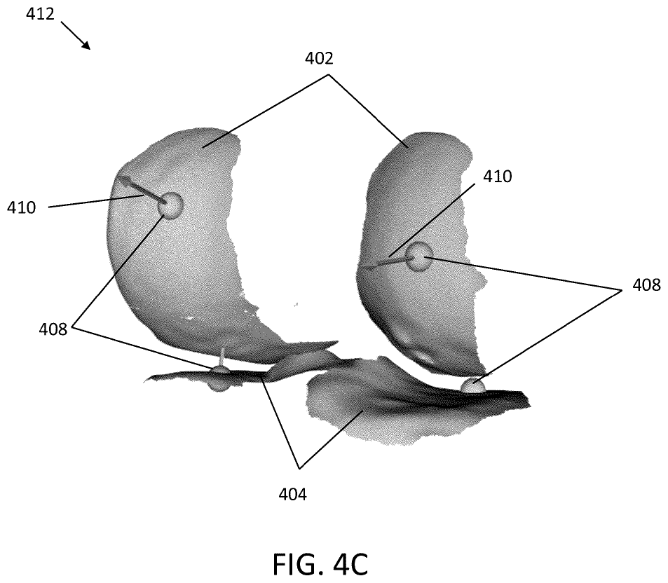

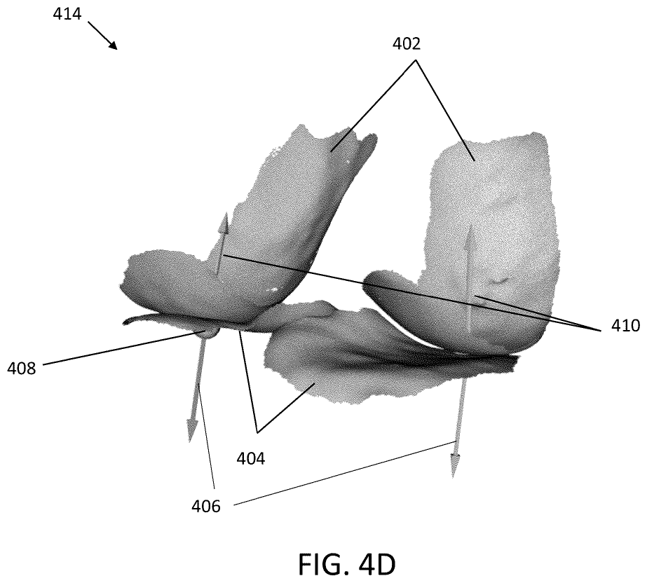

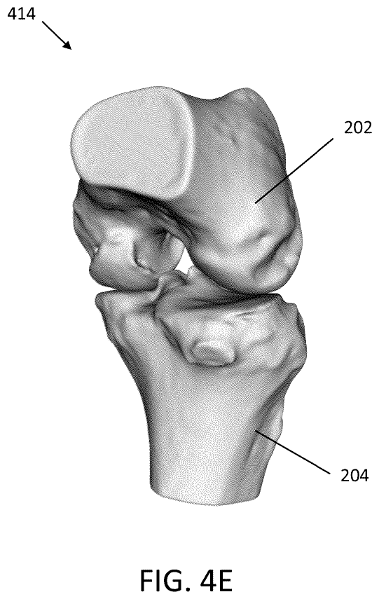

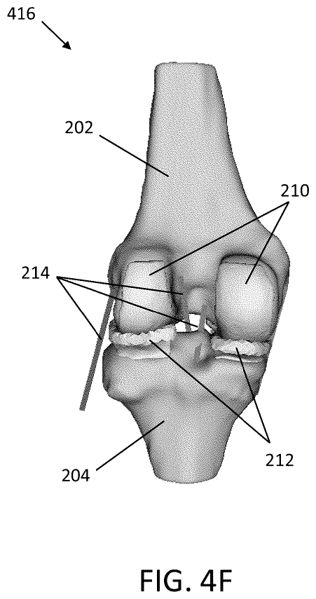

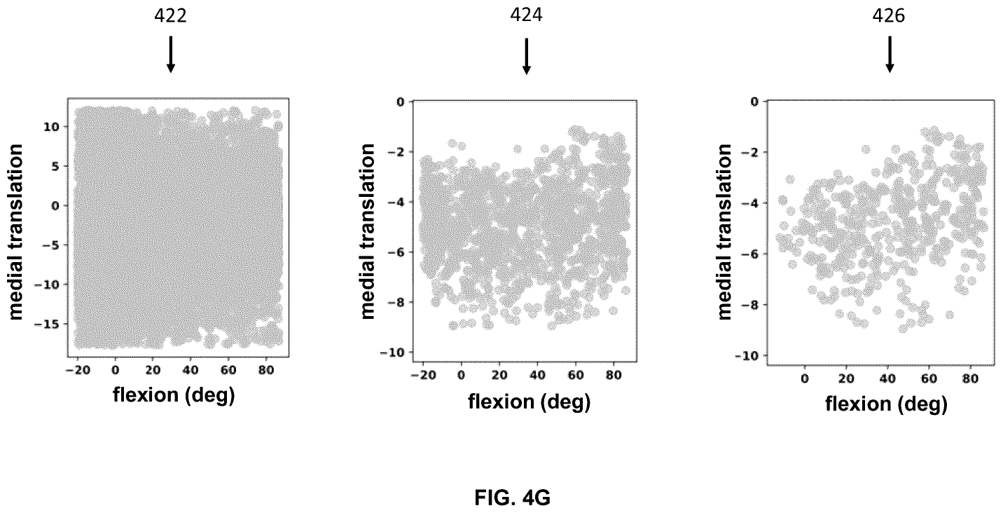

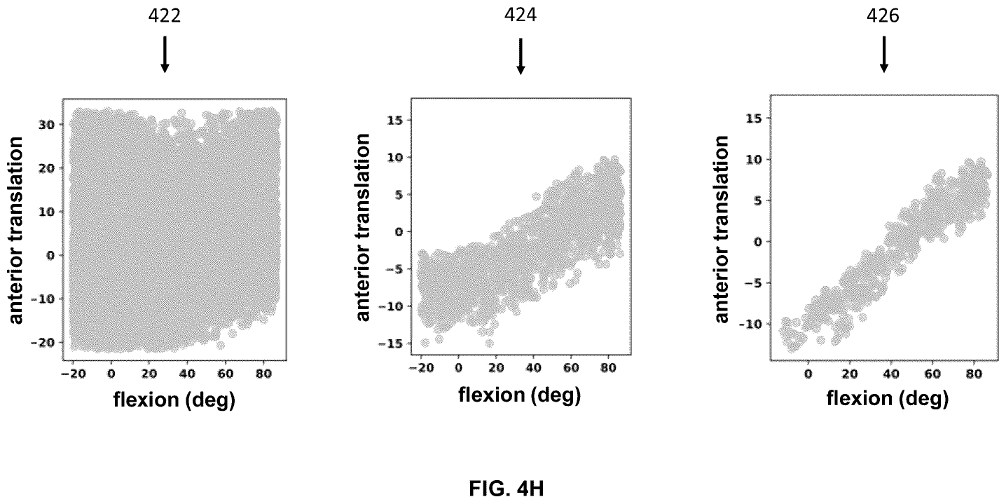

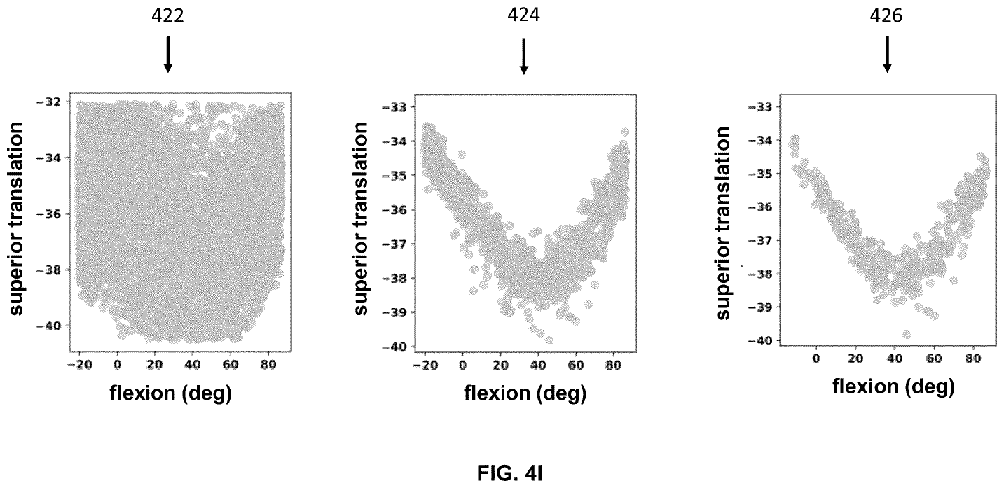

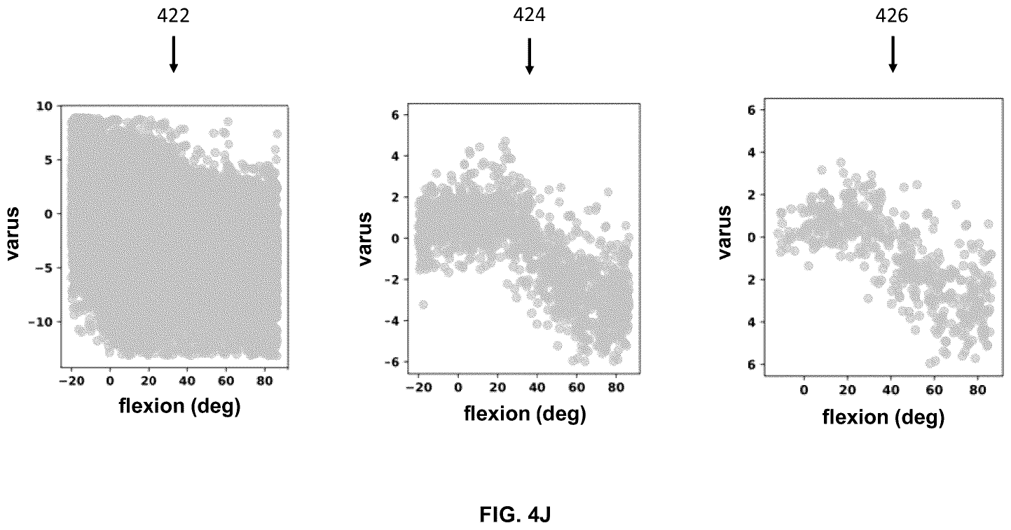

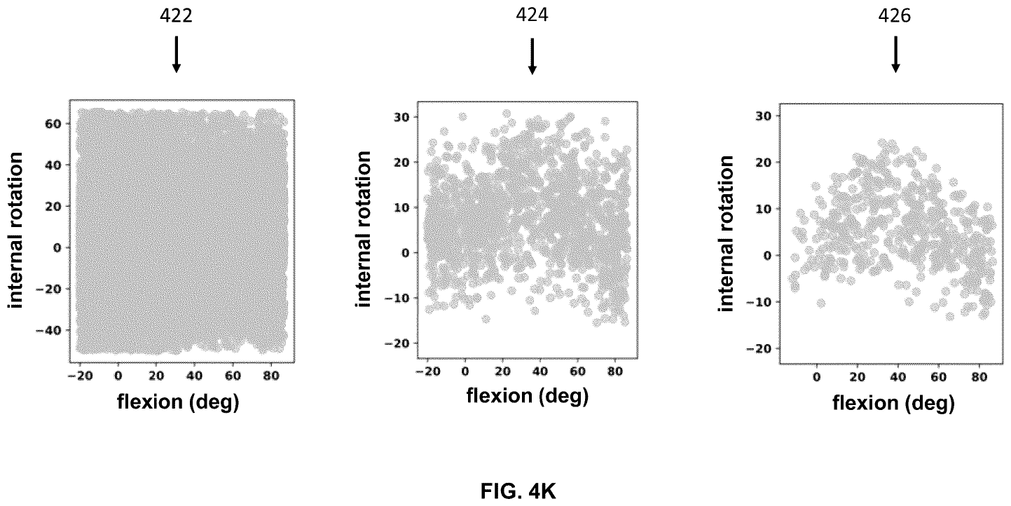

The determination of a tissue surface geometry 316 can include using the 3D volumetric data obtained in step 101 to create a usable 3D model for analysis. Specific techniques and models for such generation will be described in more detail in the discussion of FIG. 4A-4F.

Once a bone surface geometry has been generated, articular contact geometries 326 may be identified by identifying subchondral regions 328 (where the bone surface is covered by cartilage) and/or cartilage regions 330. Segmentation may similarly be applied so that subchondral bone regions, where the bone surface is covered by cartilage, may be identified. Cartilage segmentation 310 and cartilage geometry 320 can optionally be applied to determine or augment the identification of articular contact regions 326. Segmentation is useful for simplifying the bone surface geometries generated in step 318, but bone surface geometry may be determined without segmentation in some embodiments.

Generating a coordinate system 332 may permit description of joint rotations and translations in a clinically meaningful manner. Generation of the coordinate system 332 may be based on anatomical landmarks 334, which can be measured on geometries of bone 318, or alternatively on bone segmentations 308, or 3D image volume 200. Further, generating the coordinate system 332 may use optimization approaches to determine origin and axes of bone affixed coordinate systems by fits to geometry 336. A coordinate system may be generated for each bone (e.g., femur and tibia) to determine local coordinate systems on each, and/or for an entire joint (e.g., according to a kinematic convention) to commonly describe joint rotations and translations.

Referring now to FIG. 3C, a determination of contact point combinations 338 can be made by creating populations of candidate contact points on medial and lateral articular contact geometries of the femur 340 and on medial and lateral articular contact geometries of tibia 342. For each bone, databases of contact pairs across medial and lateral sides are determined for each bone followed by contact combinations of between bones 344, resulting in a database of four contact point sets, two on femur and two on tibia at medial and lateral sides.

Because this initial population may contain a large dataset of pairs that are physically feasible and pairs that are physically impossible, optional steps for reducing the population of possible contact point sets may be implemented in step 346. For the knee joint, the tibiofemoral contact occurs at medial and lateral sides. For a feasible contact between femoral and tibial articular contact surfaces, the distance between medial and lateral femoral contact points should be equal to the distance between medial and lateral tibial contact points. In addition, the angle between the normals of medial and lateral femoral contact points should be equal to the angle between medial and lateral tibial contact points. These two constraints may be used to further filter quadruples of contact points for each data entry based on set thresholds to reduce the size of the contact combination population.

Referring back to FIG. 3A, the database of candidate contact points is then used to determine a database of joint rotation and translations 348. For each data entry of two femoral and two tibial contact points on medial and lateral sides, joint rotation and translation can be determined, for example, using an optimization method that enforces rigid contact conditions. For example, to obtain rigid contact, medial contact points on femur and tibia should be coincident, lateral contact points on femur and tibia should be coincident, the normals of medial contact points on femur and tibia should be aligned (equal and opposite), and the normals of lateral contact points on femur and tibia should be aligned. It is understood that for given contact point candidates, there is no guarantee to satisfy this condition exactly. Thus, optimization allows determinations of joint translations and rotations that minimize the violation of these constraints.

Next, reducing the number of data entries 350 may further include positioning and orienting geometries relative to each other based on the given joint rotations and translations. As shown in FIG. 3D, reducing the joint translation and rotations database includes determining a signed distance of a whole femoral articular surface against a whole tibial articular surface and removing possible tibiofemoral poses and orientations that exceed a penetration threshold 352, determining a signed distance of a whole femur bone surface against a whole tibia bone surface and removing possible tibiofemoral poses and orientations that exceed a penetration threshold 354, determining a signed distance of a whole femoral cartilage surface against a whole tibial cartilage surface and removing possible tibiofemoral poses and orientations that exceed a penetration threshold 356, affixing menisci surfaces to tibia and determining a signed distance of a whole articular femur surface against menisci surfaces and removing possible tibiofemoral poses and orientations that exceed a penetration threshold 358, and/or determining ligament lengths and removing possible tibiofemoral poses and orientations where ligament lengths exceed a set threshold 360. A ligament length based filtering option may utilize calculation of ligament length distributions across the plurality of joint rotations and translations, across the base first, and then determining a threshold based on statistical metrics of each ligament's length.

The penetration threshold of steps 350 may refer to a limit that is imposed on the types of tibiofemoral poses that may be rendered as solutions. For example, a solution where one bone volume overlaps another bone volume creates a physical impossibility because it requires two bones to occupy the same space. Hence, a dividing threshold to prevent penetration can be implemented. It will be appreciated that other methods of eliminating possible tibiofemoral poses by other physical constraints relating to the mechanics of the knee and connective tissue can be further implemented.

Finally, referring back again to FIG. 3A, a resulting population set of joint rotations and translations 362 can be statistically represented as metrics of movement bounds 364. Such a population of tibiofemoral poses can be included in a report for a patient or used by clinicians to determine a knee movement capacity, which may inform diagnosis, treatment and candidacy for certain surgeries or implants.

The foregoing description to determine movement capacity can yield the results shown in FIGS. 4A-4K. More particularly, the outcomes of the steps for determining tissue geometries 316 and identifying articular contact geometries 326 may be visualized in a three-dimensional model as shown in FIG. 4A. When additional tissues are segmented as part of step 306 and their geometries are determined as part of step 316, three-dimensional model may include geometries of cartilage, menisci, ligament, and/or the like, as shown in FIG. 4F.

FIG. 4A shows an example knee surface geometry 400 represented by a triangulated surface based on manual segmentation from CT images. In this example, medial and lateral femoral contact regions 402 of the femur 202 and medial and lateral tibial contact regions 404 of the tibia 204 are shown. Surface geometries may be represented by techniques such as a point cloud or triangulated surface (also referred to as a mesh). A mesh of articular contact geometries is shown in FIG. 4B. More particularly, FIG. 4B shows the medial and lateral femoral contact regions 402 and medial and lateral tibial contact regions 404 as triangulated meshes, where a combination of contact points 408 is sampled. FIG. 4B also illustrates the normals 410 of the contact points 408. As noted above, a contact points database is generated by the combination of points of the medial and lateral femoral contact regions against the medial and lateral tibial contact regions. Alternatively, other visual renderings of the surface geometry, such as a point cloud or the like, may be used.

Different combinations of contact pairs are used to determine different poses and orientations of the knee (tibiofemoral joint kinematic configurations). As noted above, determination of joint rotations and translations 348 can be made by minimizing the distance between the femoral and tibial contact points 408 on lateral and medial sides, and by minimizing the deviation of contact point normals 410 for medial and lateral sides. This minimization allows for the approximation of joint rotations and translations for which the bone surfaces could come into contact with each other at candidate contact points.

An example of a resulting joint kinematic configuration for a sample of contact point combinations is shown in FIGS. 4C, 4D, and 4E. In FIG. 4C, a sample set of candidate contact points 408 and contact point normals 410 are shown on femoral articular contact surfaces 402 and tibial articular contact surfaces 404 for an unaligned joint 412. In FIG. 4D, a sample solution for an aligned joint 414 is shown where the femoral articular surface 402 is repositioned and re-oriented on the tibial articular surface 404 such that contact points 408 and their normals 406, 410 are aligned. The femoral contact point normal 406 and the tibial contact point normal 410 may be expressed as vectors perpendicular to the articular surfaces 402 and 404 at the sampled contact point locations, but may also be represented in other mathematical forms using, for example, angular and scalar values. FIG. 4E illustrates a reconstruction of femur 202 and tibia 204 geometries according to the sample solution illustrated in the aligned joint 414 of FIG. 4D.

The database of joint kinematic configurations is a large cohort of permutations of tibiofemoral poses, determined by possible joint translations and rotations which are restricted by rigid contact at both sides. Additionally, bone and articular contact geometries (from subchondral regions of bone surfaces or articular cartilage surfaces), and if identified, cartilage, menisci, and ligament geometries, can allow for exclusion of possible solutions of the cohort. For example, inextensibility (constant length) or rope like behavior (maximum permissible length) for ligaments can be used to identify possible range of motion boundaries, where sets outside of the boundaries do not represent possible orientations and can be discarded. Thus, the dataset can be reduced by eliminating possible solutions. Exclusion of such solutions may be based on, for example, the penetration of opposing articular contact surfaces and opposing bone surfaces, resulting in a reduced database of tibiofemoral translations and rotations.

FIG. 4F illustrates a more complete model of an example tibiofemoral joint of a knee 416 represented by triangulated surface geometries based on manual segmentation from MRI images. As illustrated, ligaments 214 and meniscus 212 can be shown in relation to the cartilage 210 and position of the femur 202 and tibia 204.

In FIGS. 4G-4K, sample solutions after filtering of the database are shown where six degrees of freedom rotations and translations of the tibiofemoral joint are visualized as point clouds across flexion angle of the joint 422 (movement capacity afforded by articular contact). These point clouds show the flexion angle in degrees (along the horizontal axes) against the medial translation (FIG. 4G), anterior translation (FIG. 4H), superior translation (FIG. 4I), varus-valgus (FIG. 4J), and internal rotation (FIG. 4K) of the joint. Further reduction of the database is possible based on the penetration of femoral contact surfaces to menisci and is visualized as point clouds 424 (movement capacity afforded by meniscal function). Additionally, the identification of ligament insertion locations can be used to impose further geometric limitations on a solution dataset and exclude possible solutions from the cohort based on ligament behavior, as shown in point clouds 426 (movement capacity afforded by ligament function). As seen in FIGS. 4G-4K, each successive limitation reduces the number of possible rotations and translations of the joint. As an example, identifying a ligament insertion location may include segmenting insertion footprints on joint bones on 3D data. These ligament insertion footprints may be determined by identifying regions of interest, for example, regions of the joint bones where tissue intensity is elevated. Additionally, the centroid of a ligament insertion location may be determined based on the segmented ligament insertion footprints. It is contemplated that other geometric determinations may be made based on the segmented ligament insertion footprints, and in turn, aid the determination of a ligament insertion location.

In the following paragraphs, methods for determining a joint-specific motion signature 103 will be described. As described above, high bone intensity regions can indicate certain tibiofemoral poses where mechanical loading is high. Accordingly, locations on articular contact surfaces where the bone exhibits high intensity can be indicative of joint rotations and translations. Motion signature may refer to preferred translations and rotations dictated by alignment of locations of high bone intensity regions under articular contact surfaces, which can also refer to habitual joint movements or tendencies.

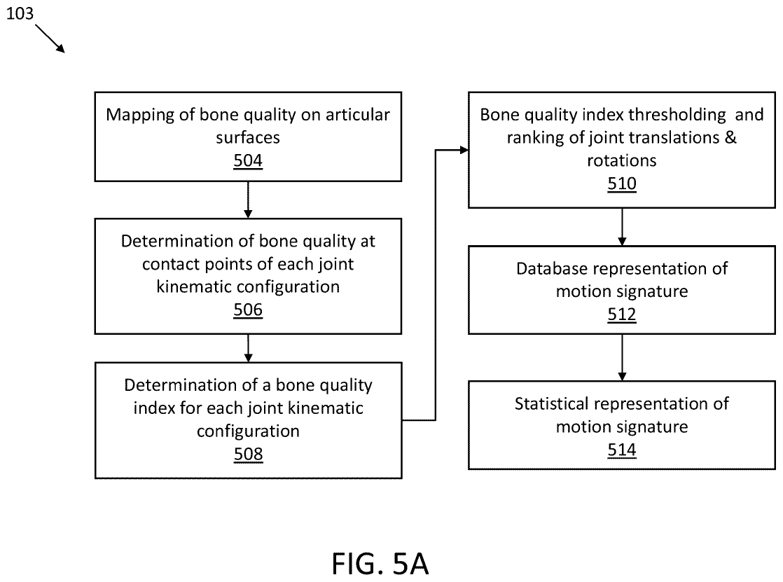

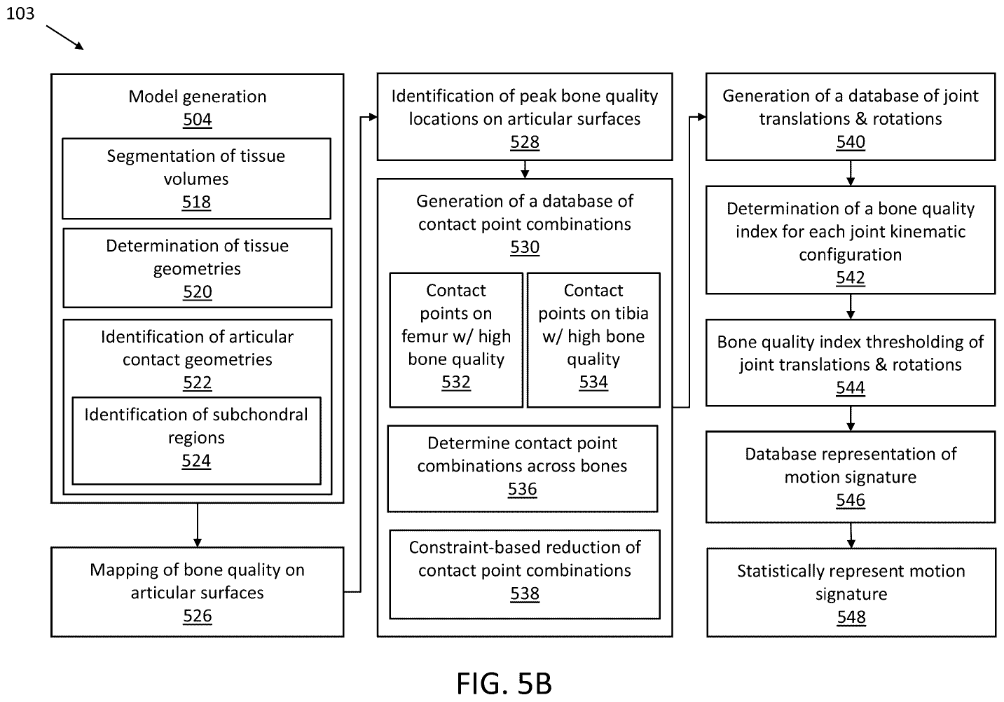

When a database of kinematic configuration exists (for example, the outcome of the method for movement capacity determination 362), elimination of solutions where contact points have relatively low bone intensities (and thus not indicative of mechanical loading) can lead to determination of motion signature 103. An example of this process for determining knee-specific motion signature is shown in FIG. 5A.

The process begins with mapping of tissue quality (for example, bone) on articular surface 504, using 3D volumetric image data 200 and articular contact geometry (e.g., as determined in step 326) as inputs. This mapping overlays articular contact geometry over an image, queries the image for every point on the surface where a bone intensity value can be transferred from the image to the geometry and recorded. This query may include overlaying a kernel volume (e.g., a prism of set size) attached to the point (e.g., kernel volume pointing inwards towards bone along the contact surface normal), sampling and averaging bone intensity where the kernel volume intersects with the image volume, and assigning the resultant scalar as a value to the point. One can use any tissue metric, image or geometry based, as tissue quality and conduct any other type of querying (e.g., using radiomics).

Databases containing previously determined joint rotations and translations 362 (and their corresponding database of contact point combinations 338) can then be used to determine bone intensities at contact points of joint kinematics configurations 506. Determination of an aggregate bone quality index for each joint kinematic configuration 508 then facilitates ranking and filtering of the database 510, based on a set threshold of the bone quality index. The resulting population set of joint rotations and translations 512 can then be statistically represented as metrics of habitual movement patterns 514. Such a population of tibiofemoral poses can be included in a report for a patient or used by clinicians to determine a knee motion signature, which may inform diagnosis, treatment and candidacy for certain surgeries or implants.

In another example embodiment of determining a joint-specific motion signature 103, the process accommodates calculation of motion signature without an existing database of joint kinematic configurations (thus when a priori determination of movement capacity does not exist). The alternative process includes the reconstruction of a bone surface geometry 520, relying on bone segmentation 518 and based on the 3D volumetric data 200 obtained from 3D imaging 101. In addition, generation of articular contact regional geometries 522 can be completed utilizing identification of subchondral regions 524. These techniques may be the same or similar to the processes described for determination of movement capacity 102; however, alternative methods may be desired for this application. Similarly, the segmentation of tissue volumes 518, determination of tissue geometries 520, identification of contact geometries 522, and identification of subchondral regions 524 may utilize the same methods described for the corresponding steps described above with respect to determining movement capacity 102.

As discussed above for the method for determining motion signature with an existing joint translation and rotation database, the method further includes a step for mapping of tissue quality (in this case bone) on articular surface 526, using 3D volumetric image data and articular contact geometry as inputs.

Next, locations of local peaks of high bone intensity on articular contact surfaces are identified 528 where the intensity value at the contact point is higher than neighboring points. Using these points as a reduced set of contact point candidates, a database of contact point combinations may be generated 530. More particularly, this determination can be made by creating populations of points with local bone intensity peaks on each the femur 532 and the tibia 534 for each contact region. For each bone, databases of contact pairs across medial and lateral sides can be determined for each bone followed by contact combinations between bones 536, resulting in a database of four contact point sets on the medial and lateral sides of the tibia and the femur.

As above, this initial population may contain a large dataset of pairs that are physically feasible and pairs that are physically impossible. Methods and techniques of reducing the size of this contact combination population 538 to only those that are physically feasible can thus be implemented. Finally, an estimation of tibiofemoral poses may be made 540 from the database of contact point combinations. As contact points are selectively extracted from regions of high bone intensity (regions of high mechanical stress or loading), the database of joint rotations and translations can be analyzed and indicate that these might be ideal candidate solutions for determining a motion signature (e.g., preferred tibiofemoral poses). As also suggested above, the generation of a database of contact point combinations 530, constraint-based reduction of the contact points in the database 538, and generation of a database of joint translations and rotations 540 may be implemented in the manner described above with respect to the corresponding steps for determining movement capacity.

Following generation of the joint translation and rotation database 540, an aggregate bone quality index for each joint kinematic configuration can be determined 542, the elements of the database may be ranked and filtered 544 based on a set threshold on the bone quality index, a final database representative of the motion signature may be generated 546, and finally a statistical representation of the resulting population set of joint rotations and translations as metrics of motion signature 548 can be generated.

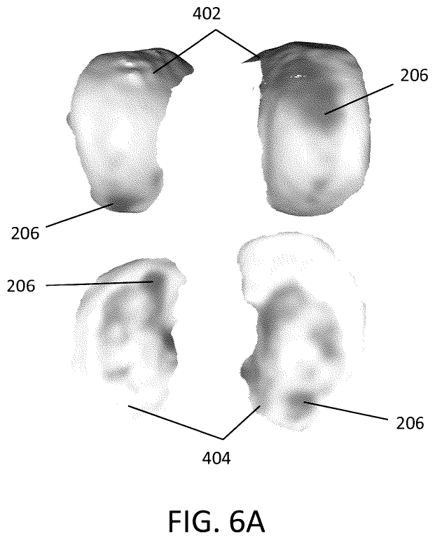

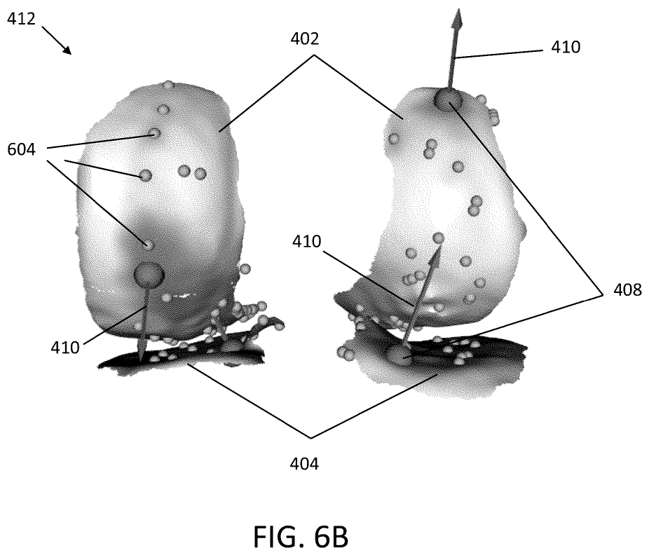

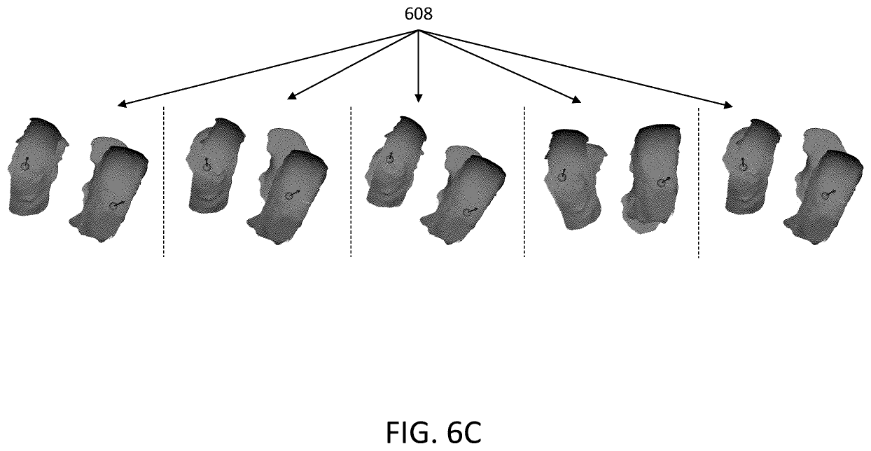

Steps that are described to determine motion signature 103 can yield the results shown in FIGS. 6A-6C. FIG. 6A illustrates a bone quality heat map over femoral 402 and tibial 404 articular contact surfaces. In some embodiments, the heat map may be coded and quantified in a variety of ways. In this example, the bone quality metric is averaged bone intensity where for each point on a subchondral region, a prismatic volume (a kernel of set size in multiples of voxel dimensions) was overlaid on the image inwards (towards the bone) along the surface normal and the bone intensity in this kernel volume can be then averaged and assigned to the point as a bone quality metric. Additionally, subchondral regions 206 can be identified from the bone quality heat map.

In FIG. 6B, an example combination of contact points 408 and contact point normals 410 is shown on femoral 402 and tibial 404 articular contact surfaces of the unaligned joint 412. This combination of contact points is selected from points on the articular surface that have a concentration of high-intensity readings shown as local peaks of bone quality region 604, indicating areas of high load transmission. The location of a global peak and all locations of local peaks of the bone quality metric can also be identified based on a mapping of bone quality such as in FIG. 6A. The reduced databased of candidate contact points (based on bone intensity local peak locations) can be used to generate contact pairs on the femur and tibia in a combinatorial fashion. In some embodiments, the heat map may be color-coded.

Tibiofemoral poses can be determined by using a minimization approach applied to the database of contact point combinations to obtain motion signature. By further solving this optimization problem for the combination of peak bone intensity locations, each combination of the candidate contact points are aligned and a large cohort of possible joint translations and rotations can be determined. FIG. 6C provides a visualization of joint kinematic configurations 608 from a cohort of possible tibiofemoral poses obtained by the alignment of peak bone intensity locations shown in FIG. 6B. Similar to the step of determining movement capacity 102, the cohort of solutions for determining motion signature may be further limited by imposing geometric rules on ligaments and penetration thresholds of articular surfaces, bones, cartilage and menisci. The motion signature can be augmented by ranking the poses and orientations to assemble them into a movement trajectory. Such ranking can range from ordering these configurations using flexion angle or weighing based on a scalar representation of tissue quality metrics (e.g., bone intensity) at corresponding tibial and femoral contact points.

Finally referring back to FIG. 1, once the movement capacity and/or motion signature are determined 102, 103, they may be output 104 as part of a report of the individualized biomechanical markers of the patient's joint. Further, the relationship between the movement capacity and motion signature can quantified as a measure of functional demand against capacity. As noted above, these biomarkers (e.g., the movement capacity and motion signature) can be used for diagnostics, clinical interventions, and the like. In addition to reporting the individualized information, normal metric values/ranges may also be illustrated on the reports. These reports may, for example, provide scalar quantifications of the metrics. These metrics may be shown as graphs, as part of a table, mapped to images of the knee, and the like. Further, models of the knee joint in various orientations may also be generated based on the metrics and illustrated on the reports. For example, any of the images in FIGS. 4A-4K and 6A-6C may be included with the report.

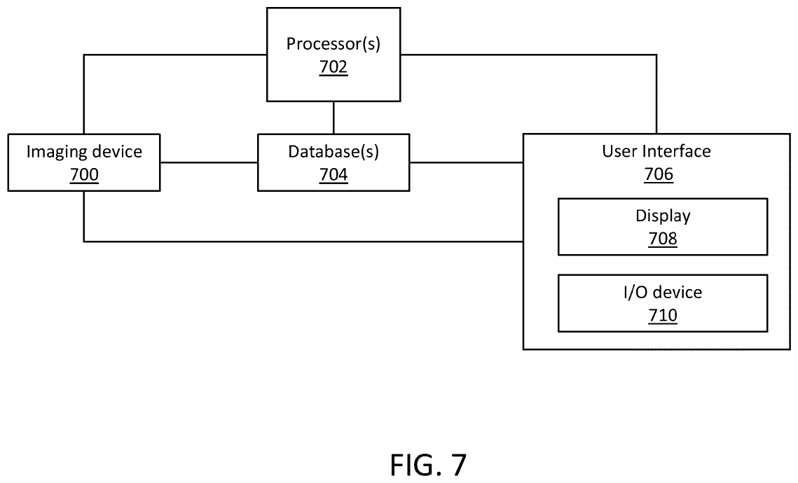

FIG. 7 is a block diagram of an example system suitable for carrying out the methods described herein. The system includes an imaging device 700, which may be a CT, MRI, or other modality as discussed above for obtaining 3D data. Further, one or more processors 702 are collectively programmed or otherwise configured to perform any of the above-described steps. For example, the one or more processors may be configured to control operation of the imaging device 700, perform any image processing and analysis, reconstruction, optimization, and the like. Furthermore, one or more processors may be configured to control and execute some or all steps for determination of movement capacity and motion signature from 3D data.

Depending on the embodiment, the 3D data may be uploaded, stored and/or retrieved from a database 704 (e.g., implemented by any type of memory, hard disk, or the like). This 3D data may further be associated in the database 704 with information about the patient, such as age, gender, medical history and the like. The database 704 may be a local storage or a remote storage, such as cloud storage. Databases of contact point candidates and joint rotations and translations, and knee-specific movement capacity and motion signature metrics may be uploaded, stored and/or retrieved from the database 704. A user may interact with the imaging device 700, one or more processors 702, and/or database(s) 704 via a user interface 706. The user interface may have, for example, a display 708 and an I/O device 710. The above-noted reports may be generated by the one or more processors 702 and provided by the user interface 706.

While the above elements of the system are shown as distinct, they may also be integrated in any manner. For example, control of the imaging device 700 may be implemented via a processor 702 associated with the imaging device 700 while other processing steps are performed by one or more separate processors 702 (e.g., as part of other computer systems). In another example, the display 708 and I/O device 710 may be integrated as a touchscreen, and may have its own control processor 702.

Additionally, it is noted that the processing described above may be provided as part of a system product or provided by remote access, for example, as a Software-as-a-Service product. Such a product may be an add-on for existing products related to surgical planning (e.g., knee implantation, soft tissue reconstruction), radiological imaging (e.g., CT acquisition and analysis), as standalone product executed on a local computer, or as a cloud-based product for on demand analysis.

While various features are presented above, it should be understood that the features may be used singly or in any combination thereof. Further, it should be understood that variations and modifications may occur to those skilled in the art to which the claimed examples pertain.

1. A method comprising:
acquiring three-dimensional (3D) data of a joint;
reconstructing an articular contact geometry of the joint based on the 3D data; and
determining movement capacity of the joint based on the reconstructed articular contact geometry, or
determining a tissue quality on articular contact geometry based on the 3D data and determining a motion signature of the joint based on the reconstructed articular contact geometry and the tissue quality.

2. The method of claim 1, wherein the joint is a musculoskeletal joint.

3. The method of claim 1, wherein the joint is an artificial joint.

4. The method of claim 1, wherein reconstructing the articular contact geometry comprises:
segmenting bones of the joint based on the 3D data;
determining subchondral regions of bone surface geometry of the joint based on the segmented bones and the 3D data; and
determining articular contact surfaces based on the subchondral regions.

5. The method of claim 1, comprising determining the movement capacity of the joint,
wherein determining the movement capacity of the joint comprises determining combinations of contact points between opposing articular contact surfaces of the reconstructed articular contact geometry,
wherein each determined combination of contact points corresponds to a pose and orientation of the joint.

6. The method of claim 5, wherein determining the movement capacity or the motion signature of the joint comprises excluding determined combinations of contact points in which opposing articular contact surfaces penetrate each other beyond a predetermined threshold.

7. The method of claim 5,
wherein each of the opposing articular contact surfaces have corresponding contact points, and
wherein alignment of the corresponding contact points is constrained based on geometric relationships between contact points of opposing articular surfaces.

8. The method of claim 1, comprising determining the tissue quality and determining the motion signature, wherein determining the motion signature comprises:
mapping the determined tissue quality on the articular contact geometry; and
estimating joint rotations and translations based on contact point sets of opposing articular contact surfaces.

9. The method of claim 8, further comprising:
excluding estimated joint rotations and translations based on thresholds of the determined tissue quality mapped to the articular contact geometry at the contact points.

10. The method of claim 8, wherein the joint rotations and translations are estimated based on locations of local peaks of the determined tissue quality mapped to the articular geometry as contact point candidates, and based on a cumulative tissue quality across contact points of each joint rotation and translation.

11. The method of claim 1, further comprising:
segmenting cartilage of the joint based on the 3D data; and
generating surface geometries of cartilage based on the segmented cartilage,
wherein reconstructing the articular contact geometry comprises generating articular surfaces based on articulating portions of cartilage determined from the segmented cartilage and generated surface geometries of the cartilage.

12. The method of claim 11, wherein determining the movement capacity or motion signature comprises:
estimating joint rotations and translations based on contact point sets of opposing articular contact surfaces; and
excluding joint rotations and translations in which cartilage surfaces penetrate each other beyond a predetermined threshold.

13. The method of claim 1, wherein the joint is a knee and the method further comprises:
segmenting menisci tissue of the knee based on the 3D data; and
generating surface geometry of menisci based on the segmented menisci,
wherein reconstructing the articular contact geometry comprises generating articular surfaces based on the segmented menisci and generated surface geometries of the menisci.

14. The method of claim 13, wherein determining the movement capacity or the motion signature comprises:
estimating joint rotations and translations based on contact point sets of opposing articular contact surfaces; and
excluding joint rotations and translations in which opposing articular contact surfaces and menisci penetrate each other beyond a predetermined threshold.

15. The method of claim 1, wherein the method further comprises determining ligament insertions by:
segmenting ligament insertion footprints on joint bones based on the 3D data; and
identifying a centroid of ligament insertion based on the segmented ligament insertion footprints, or
identifying elevated tissue intensity regions on the joint bones and estimating ligament insertion footprints based on the identified elevated tissue intensity regions.

16. The method of claim 15, wherein determining the movement capacity or motion signature comprises:
estimating joint rotations and translations based on contact point sets of opposing articular contact surfaces;
determining a length of a ligament for the estimated joint rotations and translations based on the estimated ligament insertion footprints;
determining a statistical distribution of the determined ligament lengths across a plurality of joint rotations and translations; and
excluding joint rotations and translations in which ligament lengths exceed a predetermined threshold.

17. The method of claim 1, wherein the 3D data of the joint is of an unloaded configuration of the joint.

18. The method of claim 1, wherein the 3D data of the joint is of a loaded configuration of the joint.

19. The method of claim 1, further comprising:
generating a report of the determined movement capacity and/or the determined motion signature;
outputting the report; and
diagnosing or treating a patient based on the determined movement capacity or the determined motion signature.

20. A method comprising:
acquiring computed tomography (CT) or magnetic resonance imaging (MRI) three-dimensional (3D) data of a knee of a patient;
segmenting a tibia and a femur of the knee based on the 3D data;
generating bone surface geometries of the tibia and the femur based on the segmentation and the 3D data;
determining articular contact geometries from bone surface geometries of the tibia and femur;
determining sets of contact point pairs between the tibia and the femur based on the articular contact geometries;
estimating tibiofemoral poses and orientations of the knee based on the determined contact pair sets;
determining movement capacity of the knee based on estimated tibiofemoral poses and orientations;
determining a bone intensity of the tibia and femur based on the 3D data;
mapping a bone quality of the tibia and the femur on the articular contact geometries of the tibia and femur based on the determined bone intensities of the tibia and femur, respectively;
estimating tibiofemoral poses and orientations of the knee based on the determined contact pair sets at elevated levels of mapped bone quality;
determining a motion signature of the knee based on the estimated tibiofemoral poses and orientations of the knee; and
diagnosing or treating the patient based on the determined movement capacity and motion signature of the knee.
